(19) United States
(12) Patent Application Publication
(10) Pub. No.: US 2011/0251515 A1
(43) Pub. Date: Oct. 13, 2011

(54) IMPLANTABLE PRESSURE INDICATOR WITH EXTERNAL INTERROGATION

(75) Inventors: Eric Claude Leuthardt, St. Louis, MO (US); Benjamin E. Alexander, St. Louis, MO (US); Samuel F. Wight, Los Angeles, CA (US); Jon T. Willie, St. Louis, MO (US); David D. Limbrick, St. Louis, MO (US); Guy M. Genin, St. Louis, MO (US); Lihong Wang, Creve Coeur, MO (US); Shelly Sakiyama-Elbert, Clayton, MO (US); David Allen Peters, St. Louis, MO (US)

(73) Assignee: WASHINGTON UNIVERSITY IN ST. LOUIS, St. Louis, MO (US)

(21) Appl. No.: 13/083,044

(22) Filed: Apr. 8, 2011

Related U.S. Application Data
(60) Provisional application No. 61/322,162, filed on Apr. 8, 2010.

(51) Int. Cl. A61B 5/00

(52) U.S. Cl. 600/561

(57) ABSTRACT

Observing intracranial physiology includes inserting a bladder at least partially filled with a fluid into a subdural region of interest within a subject, wherein the bladder is coupled in flow communication with a means of indicating a pressure exerted on the bladder within the region of interest. Embodiments also include non-invasively interrogating the means of indicating to determine the pressure exerted on the bladder.

### CROSS REFERENCE TO RELATED APPLICATIONS

This application claims the priority of Provisional Patent Application Ser. No. 61/322,162, entitled “IMPLANTABLE PRESSURE INDICATOR WITH EXTERNAL INTERROGATION”, which was filed on Apr. 8, 2010 and which is hereby incorporated by reference in its entirety.

### BACKGROUND

The embodiments described herein relate generally to non-invasively determining pressure in an intracranial region of interest and, more particularly, to confirmation of shunt function.

Approximately forty percent of cerebrospinal shunts fail within the first year after implantation, often resulting in hydrocephalus. In at least some subjects, symptoms of shunt failure are frequently non-specific and thus difficult to detect. Delays in diagnosis and/or treatment for such subjects may contribute to progressive health and cognitive problems. Evaluation is extremely costly, requires numerous radiographic studies, and often requires a surgical exploration of the shunt to confirm shunt malfunction. On average, there are currently 40,000 annual shunt-related surgical operations performed in the United States. This includes approximately 8,000 shunt replacement operations and approximately 18,000 shunt revision operations. With the existing systems, many subjects undergo unnecessary surgeries.

### BRIEF DESCRIPTION

In one aspect, a method is provided for observing intracranial physiology. The method includes inserting a bladder at least partially filled with a fluid into a subdural region of interest within a subject, wherein the bladder is coupled in flow communication with a means of indicating a pressure exerted on the bladder within the region of interest, and non-invasively interrogating the means of indicating to determine the pressure exerted on the bladder.

In another aspect, an apparatus is provided, including a bladder configured to be inserted into an intracranial region of interest within a subject and a meter coupled in flow communication with the bladder such that a change in a pressure exerted on the bladder within the region of interest is indicated by a fluid within the meter.

In another aspect, a system is provided for use in monitoring intracranial pressure of a subject. The system includes a pressure indicator having a bladder sized to be inserted into a subdural cavity within the subject and a pressure meter coupled in flow communication with the bladder. The pressure meter includes at least one indicator that is associated with a predetermined unit of pressure exerted on the bladder. The system also includes a diagnosis device configured to non-invasively determine a pressure exerted on the bladder based on the at least one indicator.

In yet another aspect, an implantable liquid gas interface is provided, including an enclosed system having a bladder with a fluid stored therein and configured to expand and contract based on a pressure exerted on the bladder within a region of interest of a subject. The enclosed system also includes a display system enclosed within a meter block, which is coupled in flow communication with the bladder such that the fluid flows into the meter block from the bladder upon an increase in pressure exerted on the bladder and flows out of the meter block to the bladder upon a decrease in pressure exerted on the bladder. The display system is arranged to provide a pattern that enables an operator to non-invasively determine the pressure exerted on the bladder within the region of interest.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

The embodiments described herein may be better understood by referring to the following description in conjunction with the accompanying drawings.

FIG. 1 is a top view of an exemplary liquid-gas interface device.

FIG. 2 is a side view of the liquid-gas interface device shown in FIG. 1.

FIG. 3 is a flowchart that illustrates an exemplary method for observing an internal physiology of a subject using the liquid-gas interface device shown in FIG. 1.

FIG. 4 is a top view of a burr hole created within a subject's skull for use in inserting the liquid-gas interface device shown in FIG. 1.

FIG. 5 is a side view of the burr hole shown in FIG. 4.

FIG. 6 is a diagnostic image that illustrates an exemplary image for use in determining an internal pressure.

Corresponding reference characters indicate corresponding parts throughout the drawings.

### DETAILED DESCRIPTION

Exemplary embodiments of the disclosure for use in determining and/or monitoring intracranial physiology are described herein. Embodiments described herein, and embodiments not described herein but supported by the below description, may be used with patients having hydrocephalus and requiring a cerebrospinal fluid shunt. For example, one embodiment described herein is an implantable intracranial pressure (ICP) monitor that may be non-invasively interrogated using an external ultrasound device. In some embodiments, the ICP monitor includes a fluid-filled bladder that is positioned within a subject's subdural space, and is coupled in flow communication with a chamber that is positioned outside of the subject's skull, but under the subject's scalp (e.g., in a subcutaneous space). The bladder indicates pressure within the intracranial space to the chamber using a fluid that moves through a series of channels or indicators provided within the chamber. A position of the fluid level within the chamber is indicative of the ICP. For example, in some embodiments, the fluid level within the chamber appears as a number of fluid bars that may be read using ultrasound imaging through the subject's scalp.

Non-invasive pressure monitoring, such as intracranial pressure monitoring, facilitates improving patient outcomes including, for example, preventing unnecessary surgeries, hastening necessary surgeries, and/or reducing lifetime radiation exposure to a patient. Moreover, such non-invasive pressure monitoring facilitates improving neurosurgical care by, for example, simplifying diagnostic decision-making and reducing emergency consults. Further, such non-invasive pressure monitoring may enable emergency room staff or clinical staff to evaluate a shunt in an ambulatory setting. Each of these may hasten emergency room turnover and shorten hospital stays for patients.

At least some known methods and devices have been proposed for non-invasively monitoring an internal pressure. However, such known methods and devices generally include electronic circuits that may require additional time and/or resources to implant and/or configure. For example, at least some known devices include an LC circuit having an inductor and a capacitor that, when coupled together, enables an electric current to alternate between the inductor and capacitor at the circuit's resonant frequency. However, such circuits are generally not compatible with magnetic resonance imaging and/or require a custom querying tool for use in determining the internal pressure. Another known device includes a radioactive indicator, in which pressure changes cause a change to a position of a radioactive element or a magnitude of detectable radiation emitted by the radioactive indicator. However, such devices generally expose the patient to a higher level of radiation and/or require a custom querying tool to determine the internal pressure. Another known device uses radiopaque indication, such that a change in the internal pressure changes a position or shape of a radiopaque element or fluid within the device. Still another known device includes a strain gage that measures a deflection of a diaphragm caused by a change in the internal pressure. However, such devices are generally inaccurate and require an electronic transmission be made by the device. Moreover, another known device includes a piezoresistive element having a resistance that changes based on the internal pressure. However, such a device is comparatively costly and/or requires a custom radio frequency (RF) querying tool. Another known device includes uses electromagnetic wave resonance generated within a resonant chamber. However, such a device requires costly external detection equipment.

Exemplary technical effects of the embodiments described herein, and embodiments not described herein but supported by the below description, include providing the ability to non-invasively monitor pressure in a subject's brain. From a surgical standpoint, embodiments of the disclosure are consistent with known surgical techniques, and may use an existing burr hole that is drilled at the time of shunt implantation. Moreover, embodiments of the disclosure mount easily to the subject's skull and include no moving parts. Furthermore, embodiments of the disclosure are compatible with magnetic resonance imaging machines and any other suitable imaging modality, such as commercially available ultrasound equipment, computed tomography equipment, and the like.

Although the embodiments described herein, and embodiments not described herein but supported by the description, relates to use of a liquid-gas interface device to monitor intracranial pressure and/or the functionality of an implanted shunt, other uses of the disclosure are envisioned. For example, embodiments of the liquid-gas interface device may be used for monitoring of internal pressures caused by an acute trauma, a brain tumor, and/or a stroke. In addition, embodiments of the liquid-gas interface device may be used to monitor or determine the effectiveness or impact of medical treatments that attempt to lower an internal pressure.

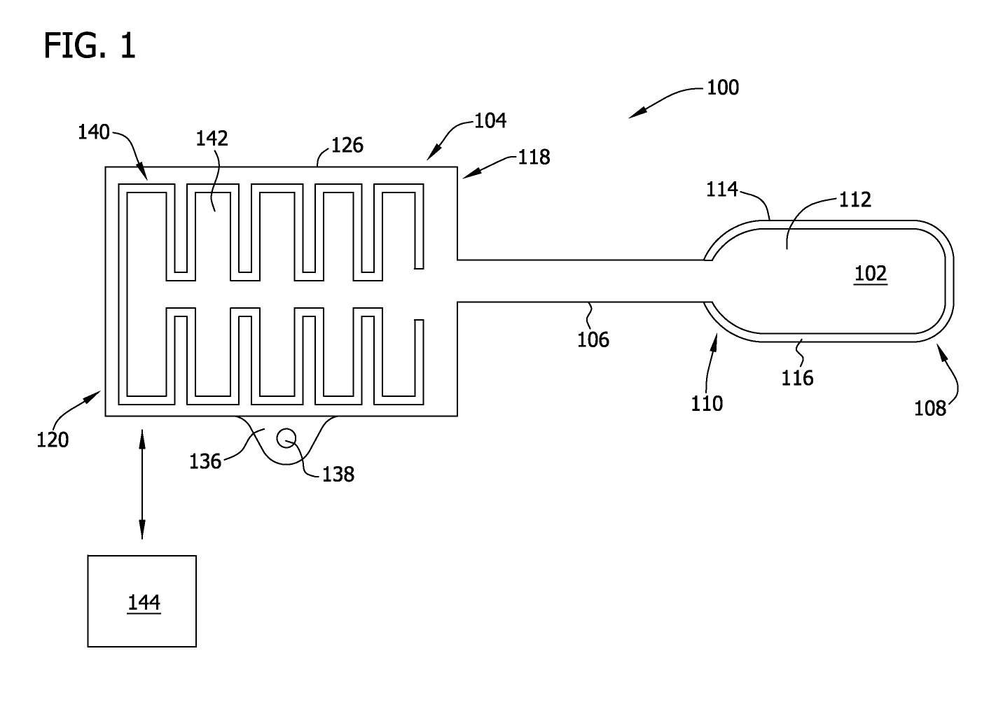

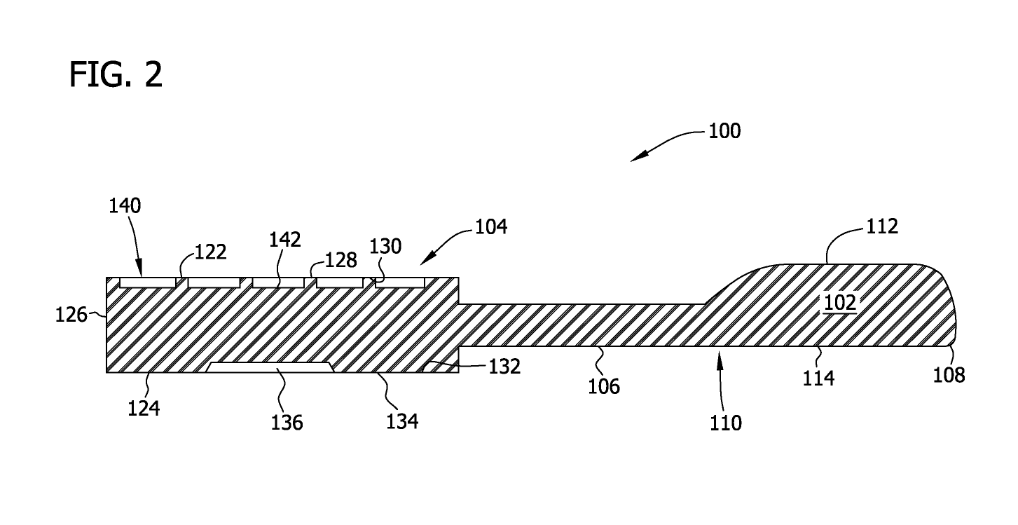

FIG. 1 is a top view of an exemplary liquid-gas interface device 100, and FIG. 2 is a side view of liquid-gas interface device 100. In the exemplary embodiment, and referring to FIGS. 1 and 2, liquid-gas interface device 100 is an enclosed system that includes a bladder 102 and a meter block 104 that is coupled to bladder 102 via a tube 106. Bladder 102 includes a first end 108 and an opposite second end 110 that is coupled in flow communication with tube 106. Moreover, bladder 102 includes a top surface 112 and an opposite bottom surface 114. Top and bottom surfaces 112 and 114 define a circumferential edge 116. In the exemplary embodiment, bladder 102 is at least partially filled with a fluid. For example, the fluid may be a saline solution or a contrast agent solution. Alternatively, the fluid may be cerebrospinal fluid from a subject. During operation, bladder 102 expands and contracts based on a pressure exerted on top surface 112 and/or bottom surface 114 within a region of interest.

In the exemplary embodiment, meter block 104 includes a first end 118 and an opposite second end 120. First end 118 is coupled in flow communication with tube 106 to facilitate fluid flow between bladder 102 and meter block 104 as described in detail below. Meter block 104 also includes a top portion 122 and an opposite bottom portion 124 with an edge 126 extending therebetween such that edge 126 couples top and bottom portions 122 and 124 about a perimeter of meter block 104. Top portion 122 includes a top surface 128 and an opposite bottom surface 130. Similarly, bottom portion 124 includes a top surface 132 and an opposite bottom surface 134. In the exemplary embodiment, top surface 128 is a top outer surface of meter block 104 and bottom surface 134 is a bottom outer surface of meter block 104. Moreover, in the exemplary embodiment, bottom portion 124 includes a flared portion 136 having an eyehole 138 extending therethrough. Flared portion 136 may be used to fix meter block 104 to a surface, such as a subject's skull. For example, a screw may be inserted through eyehole 138 to fix meter block 104 to the skull.

Moreover, in the exemplary embodiment, meter block 104 includes a means of indicating 140 a pressure, such as a pressure exerted on bladder 102 within a region of interest, such as within a subdural cavity. As shown in FIGS. 1 and 2, exemplary means for indicating 140 include a plurality of bars 142 that are oriented to indicate an incremental change in pressure on bladder 102. In the exemplary embodiment, bars 142 are oriented as shown in FIG. 1 and are coupled to bottom surface 130 as shown in FIG. 2. In an alternative embodiment, bars 142 are oriented to define a gauge. Other exemplary means for indicating 140 include, but are not limited to only including, alternative geometric patterns such as a spiral shape, a series of dots, a zigzag pattern, or another fixed pattern such as a word or series of numbers that, as the fluid fills successive characters, indicates the pressure. Additional exemplary means for indicating 140 includes, for example, a fixed portion and a movable portion that is coupled to the fixed portion at a first end such that, as additional fluid is channeled into meter block 104 from bladder 102 due to an increase in pressure, a second end of the movable portion is repositioned, such as raised. Any display system may be used as means for indicating 140 that can be readily identified with a changing air fluid level within meter block 104. Moreover, any display system may be used as means for indicating 140 such that the display system enables observation of a changing air fluid level within meter block 104 without applying electrical power to liquid-gas interface device 100. Still another exemplary means for indicating 140 includes an electrically-powered electronic display device that indicates a pressure value in a specified unit of measure, such as a number of millimeters of mercury (mmHg).

In some embodiments, liquid-gas interface device 100 is a component of a shunt device, such as a component of a shunt valve. In such an embodiment, liquid-gas interface device 100 is configured to adjust a flow of fluid through the shunt valve based on the pressure exerted on bladder 102. For example, as the pressure exerted on bladder 102 increases, liquid-gas interface device 100 adjusts the shunt valve to increase an amount or rate of fluid flow through the shunt valve.

Moreover, in some embodiments, meter block 104 includes an alarm device which is selectively activated and deactivated when a predefined amount of fluid is present within meter block 104. For example, as fluid channeled from bladder 102 into meter body 104 via tube 106, if the fluid reaches the predefined level, the alarm device is activated. The alarm device may be a light device, such as a light emitting diode (LED), which emits light in one or more colors through the subject's scalp. Alternatively or in addition, the alarm device may be a speaker device that emits a sound, a device that generates vibrations, or a device that produces any other detectable sensory output.

Referring again to FIG. 1, liquid-gas interface device 100 is externally readable using a means for observing 144. Means for observing 144 is used to non-invasively interrogate liquid-gas interface device 100 to determine an approximate pressure exerted on bladder 102. In the exemplary embodiment, means for observing 144 includes an imaging modality. Specifically, in the exemplary embodiment, means for observing 144 includes an ultrasound imaging apparatus. Alternatively, the imaging modality may be a magnetic resonance imaging (MRI) apparatus, a computed tomography (CT) imaging apparatus, a photoacoustic imaging apparatus, a plain radiography apparatus, a near infrared (NIR) apparatus, and the like. In another alternative embodiment, means for observing 144 is a remote computer. In such an embodiment, liquid-gas interface device 100 transmits a fluid level, a pressure value, and/or a change in a pressure value to the remote computer via wired or wireless communications.

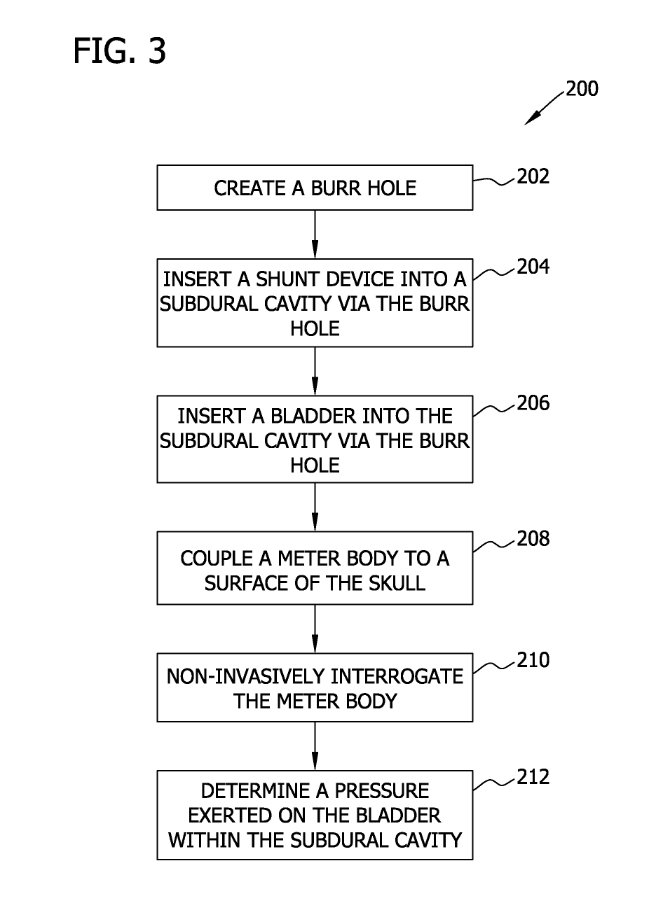

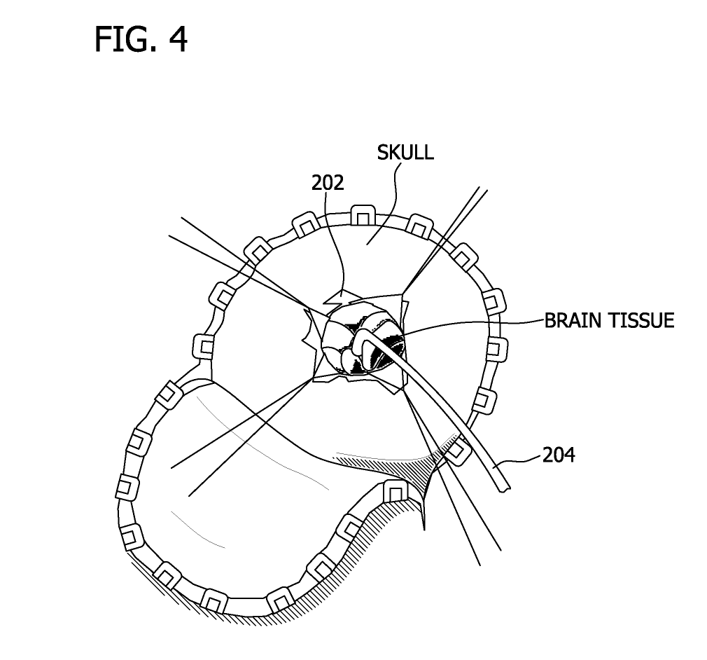

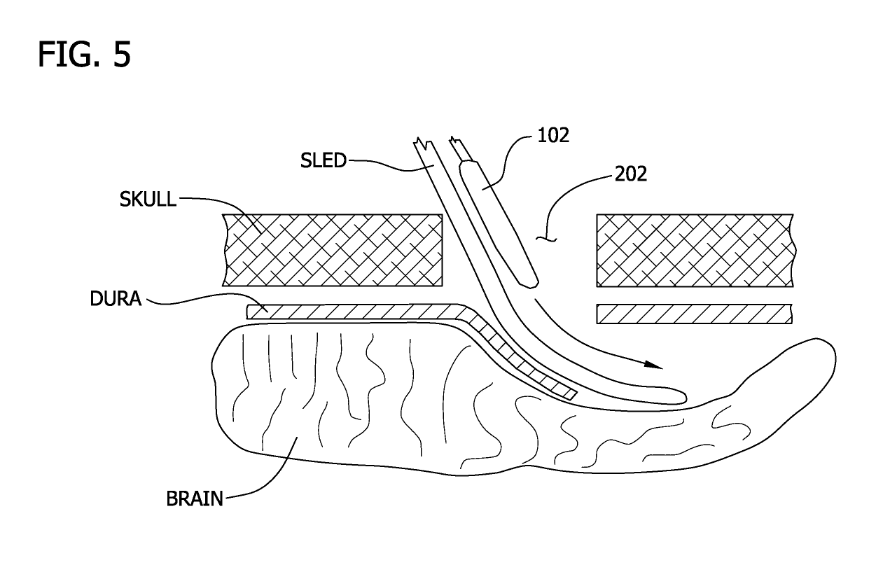

FIG. 3 is a flowchart 200 that illustrates an exemplary method for observing an internal physiology of a subject, such as intracranial physiology, and/or for monitoring an internal pressure of the subject, such as intracranial pressure. In the exemplary embodiment, a burr hole is created 202 within the subject's skull. A shunt component, is inserted 204 into the subject's brain via the burr hole, and bladder 102 (shown in FIGS. 1 and 2) is then inserted 206 into a subdural cavity via the burr hole. FIG. 4 is a top view of a burr hole created within a subject's skull, and FIG. 5 is a side view of the burr hole within the skull. As shown in FIGS. 4 and 5, a shunt component, such as a tube, is inserted into the brain via the burr hole using a sled. Bladder 102 is also inserted into the brain via the burr hole using the sled. Specifically, bladder 102 is inserted into the subdural cavity between the subject's dura and brain. Referring again to FIG. 3, and in the exemplary embodiment, meter body 104 (shown in FIGS. 1 and 2) is coupled 208 to a top surface of the skull via flare portion 136 (shown in FIGS. 1 and 2).

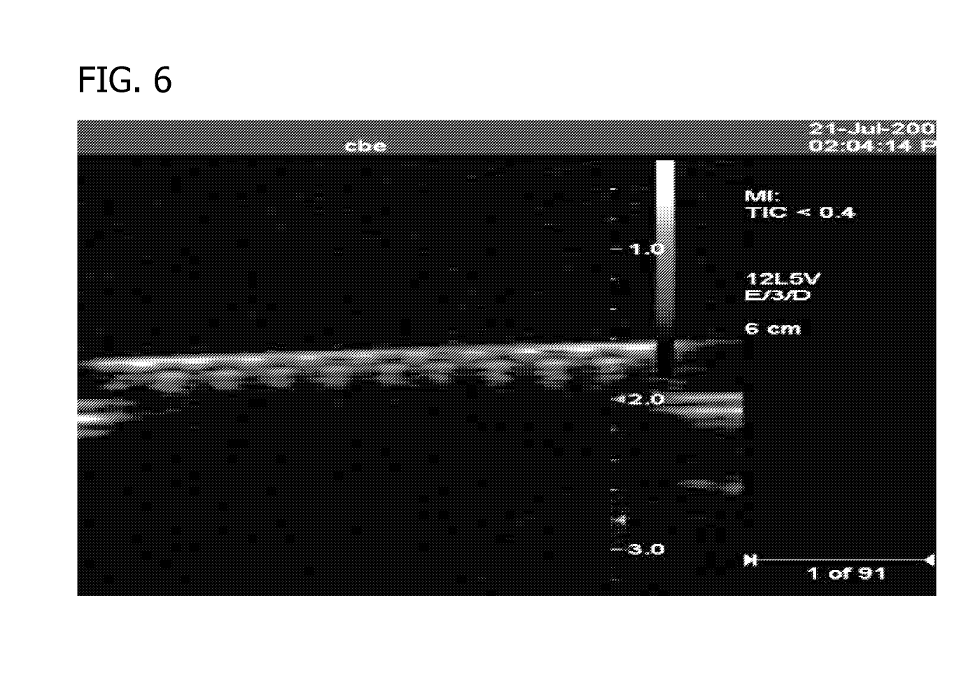

Meter body 104 is interrogated 210, such as non-invasively interrogated, to determine a pressure exerted on bladder 102. More specifically, means for observing 144 (shown in FIG. 1) is used to interrogate means for indicating 140. In the exemplary embodiment, an imaging modality, such as an ultrasound apparatus, is used to generate an image of meter block 104. The image displays a number of bars 142 (shown in FIG. 1) that correspond with a fluid level within meter body 104. An operator then determines 212 a pressure, such as an intracranial pressure, being exerted on bladder 102 based on the number of bars 142 displayed in the image. FIG. 6 is a diagnostic image that illustrates an exemplary image for use in determining the pressure exerted on bladder 102. In alternative embodiments, other imaging modalities may be used to generate the image, such as an MRI apparatus, a CT imaging apparatus, a plain radiography apparatus, an NIR apparatus, or a photoacoustic imaging apparatus. In another alternative embodiment, liquid-gas interface device 100 transmits, such as electronically transmits, a signal indicative of the pressure to a computer.

In some embodiments, liquid-gas interface device 100 is calibrated to account for a known initial pressure, an altitude of the site of use that may affect operation, and the like. For example, prior to insertion of liquid-gas interface device 100, an operator may note or record an initial pressure reading as displayed via means for indicating 140. Thereafter, the pressure exerted on bladder 102 is determined based in part on the initial pressure reading. As an example, the pressure exerted on bladder 102 may be determined by adjusting for the initial pressure, such as by subtracting the initial pressure reading from the pressure indicated by means for observing 144.

Standard medical practices that facilitate medical operative procedures may be used in connection with embodiments of the disclosure. For example, without limitation, portions and/or components of the shunt device, such as liquid-gas interface device 100, may be covered with materials such as parylene to mitigate a potential for gas leakage. Also, for example, fiduciary markers may be used to facilitate activities that use medical imaging, including, without limitation, aiding location of bladder 102 during insertion 206 of bladder 102 into the subdural cavity via the burr hole.

Exemplary embodiments of the disclosure for use in determining and/or monitoring intracranial physiology are described above in detail. The disclosure is not limited to the specific embodiments described herein but, rather, operations of the methods and/or components of the system and/or apparatus may be utilized independently and separately from other operations and/or components described herein. Further, the described operations and/or components may also be defined in, or used in combination with, other systems, methods, and/or apparatus, and are not limited to practice with only the systems, methods, and/or apparatus as described herein.

The order of execution or performance of the operations in the embodiments of the disclosure illustrated and described herein is not essential, unless otherwise specified. That is, the operations may be performed in any order, unless otherwise specified, and embodiments of the disclosure may include additional or fewer operations than those disclosed herein. For example, it is contemplated that executing or performing a particular operation before, contemporaneously with, or after another operation is within the scope of aspects of the disclosure.

In some embodiments, the term “processor” refers generally to any programmable system including systems and microcontrollers, reduced instruction set circuits (RISC), application specific integrated circuits (ASIC), programmable logic circuits (PLC), and any other circuit or processor capable of executing the functions described herein. The above examples are exemplary only, and thus are not intended to limit in any way the definition and/or meaning of the term processor.

Embodiments of the disclosure may be described in the general context of computer-executable instructions, such as program modules, executed by one or more computers or other devices. The computer-executable instructions may be organized into one or more computer-executable components or modules. Generally, program modules include, but are not limited to, routines, programs, objects, components, and data structures that perform particular tasks or implement particular abstract data types. Aspects of the disclosure may be implemented with any number and organization of such components or modules. For example, aspects of the disclosure are not limited to the specific computer-executable instructions or the specific components or modules illustrated in the figures and described herein. Other embodiments of the disclosure may include different computer-executable instructions or components having more or less functionality than illustrated and described herein.

Aspects of the disclosure transform a general-purpose computer into a special-purpose computing device when configured to execute the instructions described herein.

The computer-executable instructions may be stored on one or more computer readable media. By way of example and not limitation, computer readable media comprise computer storage media and communication media. Computer storage media store information such as computer readable instructions, data structures, program modules or other data. Communication media typically embody computer readable instructions, data structures, program modules, or other data in a modulated data signal such as a carrier wave or other transport mechanism and include any information delivery media. Combinations of any of the above are also included within the scope of computer readable media.

When introducing elements of aspects of the disclosure or embodiments thereof, the articles “a,” “an,” “the,” and “said” are intended to mean that there are one or more of the elements. The terms “comprising,” including,” and “having” are intended to be inclusive and mean that there may be additional elements other than the listed elements.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for observing intracranial physiology, the method comprising:
inserting a bladder at least partially filled with a fluid into a subdural region of interest within a subject, wherein the bladder is coupled in flow communication with a means of indicating a pressure exerted on the bladder within the region of interest;
non-invasively interrogating the means of indicating to determine the pressure exerted on the bladder.

2. A method in accordance with claim 1, wherein inserting a bladder comprises:
forming a burr hole;
inserting a shunt component through the burr hole; and
inserting the bladder through the burr hole.

3. A method in accordance with claim 1, wherein inserting a bladder comprises forming a burr hole and inserting a component of a shunt device that includes a valve.

4. A method in accordance with claim 3, wherein the means for indicating is a component of the valves, the method further comprising adjusting a flow of a fluid the valve based on the pressure exerted on the bladder.

5. A method in accordance with claim 1, wherein interrogating the means of indicating comprises:
using an imaging modality to scan the means of indicating to determine the pressure exerted on the bladder; and
generating an image that indicates the pressure exerted on the bladder.

6. An apparatus comprising:
a bladder configured to be inserted into an intracranial region of interest within a subject; and
a meter coupled in flow communication with the bladder such that a change in a pressure exerted on the bladder within the region of interest is indicated by a fluid within the meter.

7. An apparatus in accordance with claim 6, wherein the bladder is sized to be inserted through a burr hole into a subdural cavity of the subject, and wherein the burr hole is sized to receive the bladder and a shunt component.

8. An apparatus in accordance with claim 6, wherein the change in pressure exerted on the bladder within the region of interest is indicated by the fluid within the meter without applying electrical power to the apparatus.

9. An apparatus in accordance with claim 6, wherein the meter is sized to be positioned between the subject's skull and the subject's scalp.

10. An apparatus in accordance with claim 6, wherein the meter is externally readable using an imaging modality.

11. An apparatus in accordance with claim 6, wherein the meter comprises a means for indicating the pressure exerted on the bladder based on an amount of the fluid within the meter.

12. An apparatus in accordance with claim 11, wherein the means for indicating comprises at least one geometric shape that is associated with a known pressure value.

13. An apparatus in accordance with claim 6, wherein the meter comprises an alarm configured to be selectively activated and deactivated based on a level of the fluid within the meter.

14. An apparatus in accordance with claim 6, wherein the meter is coupled to a valve, the meter configured to adjust a flow enabled by the valve based on the change in pressure.

15. An apparatus in accordance with claim 6, wherein the meter is configure to communicate at least one of the change in pressure and a constant pressure to a remote diagnosis unit.

16. A system for use in monitoring intracranial pressure of a subject, the system comprising:
a pressure indicator comprising:
a bladder sized to be inserted into a subdural cavity within the subject; and
a pressure meter coupled in flow communication with the bladder, the pressure meter comprising at least one indicator that is associated with a predetermined unit of pressure exerted on the bladder; and
a diagnosis device configured to non-invasively determine a pressure exerted on the bladder based on the at least one indicator.

17. A system in accordance with claim 16, wherein the bladder is sized to be inserted into the subdural cavity through a burr hole that is sized to receive the bladder and a shunt component, and wherein the bladder has a predetermined pattern of fiduciary marks thereon to facilitate insertion of the bladder.

18. A system in accordance with claim 16, wherein the at least one indicator comprises a plurality of indicators each associated with a predetermined unit of pressure exerted on the bladder, and wherein the plurality of indicators are arranged in a geometric pattern.

19. A system in accordance with claim 16, wherein the diagnosis device comprises an imaging modality.

20. A system in accordance with claim 16, wherein the bladder is configured to expand and contract based on a pressure exerted on the bladder within a region of interest of a subject, wherein the bladder stores a fluid, and wherein the system is an enclosed system further comprising:
a display system enclosed within a meter block, the meter block coupled in flow communication with the bladder such that the fluid flows into the meter block from the bladder upon an increase in pressure exerted on the bladder and flows out of the meter block to the bladder upon a decrease in pressure exerted on the bladder, wherein the display system is arranged to provide a pattern that enables an operator to non-invasively determine the pressure exerted on the bladder within the region of interest.
